(19) United States
(12) Patent Application Publication
(10) Pub. No.: US 2026/0368536 A1
(43) Pub. Date: Nov. 19, 2026

(54) METHOD, PROGRAM, AND DEVICE FOR ACQUIRING USER-CUSTOMIZED NEURAL NETWORK MODEL FOR IDENTIFYING BIOLOGICAL INFORMATION

(71) Applicant: Medical AI Co., Ltd., Seoul (KR)

(72) Inventor: Joonmyoung Kwon, Seoul (KR)

(21) Appl. No.: 19/477,754

(22) PCT Filed: Apr. 25, 2024
(86) PCT No.: PCT/KR2024/005625

§ 371 Date: Oct. 22, 2025

(30) Foreign Application Priority Data
Apr. 25, 2023 (KR) 10-2023-0054216
May 30, 2023 (KR) 10-2023-0069381
Apr. 25, 2024 (KR) 10-2024-0055172

(51) Int. Cl. G06N 3/09; G16H 50/30

(52) U.S. Cl. CPC G06N 3/09; G16H 50/30

(57) ABSTRACT

According to one embodiment of the present disclosure, there is disclosed a method of acquiring a user-customized neural network model that identifies bio-information, the method including: predicting bio-information related to the status of a user by inputting a biosignal, detected from the user, to a pre-trained neural network model; and, when new data for the user is acquired, updating the pre-trained neural network model to be personalized based on the acquired new data; wherein the new data includes bio-information measured from the user related to the status.

### TECHNICAL FIELD

The present disclosure relates to deep learning technology in the medical field, and more particularly, to a method, program, and device for acquiring a user-customized neural network model that identifies bio-information.

### BACKGROUND ART

With the recent development of information and communication technology, deep learning technology is being utilized in various fields. In particular, deep learning technology is also being used in a variety of ways in the medical field, which has traditionally relied on specialized and limited personnel such as doctors, researchers, etc. to diagnose diseases of patients. For example, an example thereof is a case where the bio-data acquired from a patient in real time is input to a pre-trained deep learning model to analyze the status or disease of the patient.

Meanwhile, a deep learning model used in the medical field is pre-trained based on pre-set training data and provided to individual users. Accordingly, in real-world applications, a problem arises in that the deep learning model may sometimes produce different results for individual users or subjects (e.g., patients). The reason for this is that even bio-information for the same type or status may be acquired in different forms or as different values for individual users. This is also caused by the biological characteristics of individual users. Therefore, there is a demand for a scheme for personalizing even the same deep learning model for each user by reflecting the environment and characteristics of the user therein.

### DISCLOSURE

### Technical Problem

The present disclosure has been conceived in response to the above-described background art, and an object of the present invention is to provide a method, program, and device for acquiring a user-customized neural network model that identifies bio-information.

However, the objects to be achieved in the present disclosure are not limited to the object mentioned above, and other objects not mentioned may be clearly understood based on the following description.

### Technical Solution

According to one embodiment of the present disclosure for achieving the above-described object, there is disclosed a method of acquiring a user-customized neural network model that identifies bio-information, the method being performed by a computing device including at least one processor, the method including: predicting bio-information related to the status of a user by inputting a biosignal, detected from the user, to a pre-trained neural network model; and, when new data for the user is acquired, updating the pre-trained neural network model to be personalized based on the acquired new data; wherein the new data includes bio-information measured from the user related to the status.

Alternatively, the updating includes calibrating the pre-trained neural network model to be personalized based on the measured bio-information and the biosignal detected to correspond to the measured bio-information.

Alternatively, the calibration includes performing supervised learning based on the bio-information measured from the user and the biosignal detected to correspond to the bio-information measured from the user.

Alternatively, the updating includes: when new data for the user is acquired, accumulating the acquired new data into a dataset accumulated and acquired at a previous time, and updating the neural network model to be personalized based on the dataset, and the dataset includes a plurality of pieces of bio-information accumulated and measured at the previous time from the user.

Alternatively, the method includes identifying bio-information of the user, and transmitting the new data to a computing device of another user having bio-information similar to the identified bio-information so that the computing device of the other user is updated based on the new data.

Alternatively, the biosignal includes an electrocardiogram (ECG) signal, and the bio-information includes left ventricular ejection fraction (EF).

Alternatively, the method includes, when the left ventricular ejection fraction is lower than a preset value, outputting information warning of heart failure with reduced left ventricular ejection fraction of the user.

Alternatively, the updating includes monitoring the trend of the predicted bio-information, and, when a change in the predicted bio-information is detected, updating the pre-trained neural network model to be personalized based on the acquired new data.

Alternatively, the updating includes, when it is identified that a preset time has elapsed since the previous update time, updating the pre-trained neural network model to be personalized based on the acquired new data.

Alternatively, the updating step includes, when the number of times bio-information related to the status of the user has been predicted since the previous update time is equal to or larger than a preset value, updating the pre-trained neural network model to be personalized based on the acquired new data.

According to one embodiment of the present disclosure for achieving the above-described object, there is disclosed a computer program stored in a computer-readable storage medium, the computer program causing operations for acquiring a user-customized neural network model that identifies bio-information to be performed when executed by at least one processor, wherein the operations include operations of: predicting bio-information related to the status of a user by inputting a biosignal, detected from the user, to a pre-trained neural network model; and, when new data for the user is acquired, updating the pre-trained neural network model to be personalized based on the acquired new data; wherein the new data includes bio-information measured from the user related to the status.

According to one embodiment of the present disclosure for achieving the above-described object, there is disclosed a computing device for acquiring a user-customized neural network model that identifies bio-information, the computing device including: memory including program codes; a network unit; a sensing unit configured to detect bio-information of a user; and a processor configured to predict bio-information related to the status of the user by inputting a biosignal, detected from the user, to a pre-trained neural network model, and to, when new data for the user is acquired, update the pre-trained neural network model to be personalized based on the acquired new data; wherein the new data includes bio-information measured from the user related to the status.

### Advantageous Effects

According to the method of acquiring a user-customized neural network model that identifies bio-information according to one embodiment of the present disclosure, it may be possible to obtain a neural network model that identifies more accurate bio-information for a user by reflecting the characteristics of the user therein.

In addition, it may be possible to personalize a neural network model stored in a computing device to suit a user simply by acquiring bio-information for the user.

### DESCRIPTION OF DRAWINGS

FIG. 1 is an exemplary diagram of a device for acquiring a user-customized neural network model that identifies bio-information according to one embodiment of the present disclosure;

FIG. 2 is a block diagram of a computing device according to one embodiment of the present disclosure;

FIG. 3 is a flowchart schematically showing a method of acquiring a user-customized neural network model that identifies bio-information according to one embodiment of the present disclosure;

FIG. 4 is an exemplary diagram showing updating a neural network model based on first and second bio-information according to one embodiment of the present disclosure;

FIG. 5 is a flowchart showing updating a neural network model based on first and second bio-information according to one embodiment of the present disclosure;

FIG. 6 is an exemplary diagram showing updating the neural network model based on first bio-information and a plurality of pieces of second bio-information included in a dataset according to one embodiment of the present disclosure; and

FIG. 7 is a block diagram of a computing device according to another embodiment of the present disclosure.

### MODE FOR INVENTION

Embodiments of the present disclosure will be described in detail below with reference to the accompanying drawings so that those having ordinary skill in the art of the present disclosure (hereinafter, those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Furthermore, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.”

The term “N-th (N is a natural number)” used herein can be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

The term “acquiring” used herein may be understood to mean not only receiving data over a wired/wireless communication network connecting with an external device or a system, but also generating data in an on-device form.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Furthermore, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “model” used herein may be understood as a system implemented using mathematical concepts and language to solve a specific problem, a set of software units intended to solve a specific problem, or an abstract model for a process intended to solve a specific problem. For example, a neural network “model” may refer to an overall system implemented as a neural network that is provided with problem-solving capabilities through training. In this case, the neural network may be provided with problem-solving capabilities by optimizing parameters connecting nodes or neurons through training. The neural network “model” may include a single neural network, or a neural network set in which multiple neural networks are combined together.

The “data” used herein may include an “image,” a signal, and the like. The term “image” used herein may refer to multidimensional data composed of discrete image elements. In other words, the “image” may be understood as a term referring to a digital representation of an object that is visible to the human eye. For example, the term “image” may refer to multidimensional data composed of elements corresponding to pixels in a two-dimensional image. The term “image” may refer to multidimensional data composed of elements corresponding to voxels in a three-dimensional image.

The foregoing descriptions of the terms are intended to help to understand the present disclosure. Accordingly, it should be noted that unless the above-described terms are explicitly described as limiting the content of the present disclosure, the terms in the content of the present disclosure are not used in the sense of limiting the technical spirit of the present disclosure.

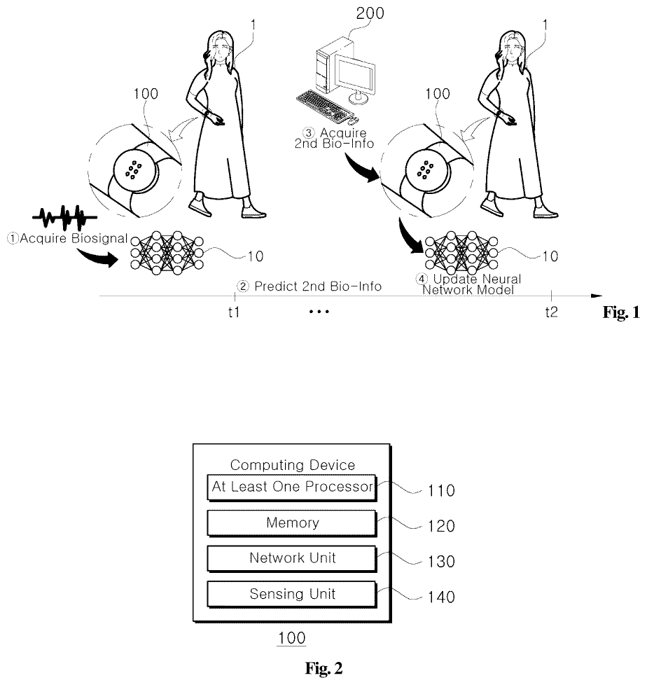

FIG. 1 is an exemplary diagram of a device for acquiring a user-customized neural network model 10 that identifies bio-information according to one embodiment of the present disclosure.

A computing device 100 according to one embodiment of the present disclosure may be a hardware device or a part of a hardware device that performs the comprehensive processing and computation of data, or may be a software-based computing environment connected over a communication network. For example, the computing device 100 may be a server that is a main agent for performing an intensive data processing function and sharing resources, or may be a client that shares resources through interaction with a server. Alternatively, the computing device 100 may be a cloud system in which multiple servers and clients comprehensively process data while interacting with each other. Since the above description is only one example related to the type of computing device 100, the type of computing device 100 may be configured in various manners within a range understandable to those skilled in the art based on the content of the present disclosure.

Referring to FIG. 1, a computing device 100 according to an embodiment of the present disclosure may be implemented as a smartwatch. However, the computing device 100 is not limited thereto, but may be implemented as various electronic devices such as a desktop, a laptop, a smartphone, a server device, a smart band, a smart ring, or the like. Furthermore, the computing device 100 may also be implemented as a biosignal measurement device that measures bio-information of a user 1 (e.g., a patient, or the like). For the purpose of understanding the present disclosure, the following description will be given on the assumption that the computing device 100 is a smartwatch.

Meanwhile, the pre-trained neural network model 10 may be stored in the computing device 100. The neural network model 10 may be a model pre-trained to identify the status, symptom, disease, bio-information, and/or the like of the user 1. In particular, the neural network model 10 may be pre-trained in accordance with the type, purpose of use, and/or the like of the computing device 100.

According to one embodiment of the present disclosure, the neural network model 10 may be pre-trained to identify another piece of bio-information of the user 1 based on the bio-information acquired from the user 1 by the computing device 100.

In this case, the bio-information may include various biosignals (e.g., an electrocardiogram (electrocardiogram) signal, an electroencephalogram (EEG) signal, an photoplethysmogram (PPG) signal, arterial blood pressure (ABP), and central venous pressure (CVP)) acquired from the user 1. Furthermore, the bio-information may include a disease (e.g., heart failure, or the like) and the possibility of the disease identified for the user 1 and a measured value (e.g., body temperature, blood pressure, left ventricular ejection fraction (EF), and/or the like) indicating the status of the user 1.

In the following description, for convenience of description of the present disclosure, the bio-information acquired by the computing device 100 and input to the neural network model 10 will be referred to as first bio-information, and the data (i.e., output data) output through the neural network model 10 by using the acquired biosignal as input data will be referred to as second bio-information.

According to one embodiment of the present disclosure, the first bio-information and the second bio-information may be different types of bio-information. The user 1 may determine the status of the user 1 by using the first bio-information and the second bio-information. As an example, the first bio-information may be an electrocardiogram (electrocardiogram) signal, and the second bio-information may be left ventricular ejection fraction. Through this, the user 1 may monitor left ventricular ejection fraction and examine the disease and health status of the user 1 in real time simply by using the computing device 100 without visiting a specialized medical institution. In particular, the disease and health status of the user 1 may be related to the heart of the user 1.

As another example, the first bio-information may include bio-information acquired from the user 1 by using a non-invasive measurement method. As an example, the first bio-information may include an electrocardiogram signal, an electroencephalogram signal, a photoplethysmogram signal, and the like. In contrast, the second bio-information may include the bio-information acquired from the user 1 by using an invasive measurement method or the bio-information acquired through direct measurement by a medical professional. As an example, the second bio-information may include body temperature, blood pressure (e.g., arterial blood pressure, central venous blood pressure, and/or the like), left ventricular ejection fraction, and/or the like. That is, the neural network model 10 may be trained to identify the second bio-information acquired based on a non-invasive method which requires the intervention of a medical professional or by which it is generally difficult to measure information by using the first bio-information acquired based on a non-invasive method by which it is easy to measure information from the user 1. Through this, the user 1 may monitor the second bio-information and examine the status of the user 1 in real time simply by using the computing device 100 without visiting a specialized medical institution.

Meanwhile, the neural network model 10 may be implemented as a multi-layer perceptron (MLP), a convolutional neural network (CNN), a recurrent neural network (RNN), a generative adversarial network, or the like. In particular, the neural network model 10 may include a plurality of residual blocks configured to extract latent features of the input first bio-information, and a classifier configured to classify second bio-information or a specific disease based on the extracted latent features.

Meanwhile, the computing device 100 may be manufactured with a neural network model 10 stored therein. Alternatively, the computing device 100 may acquire the neural network model 10 from an external computing device (e.g., a server device, or the like) that operates in conjunction with the computing device 100. In this case, the computing device 100 may update the neural network model 10 stored therein to suit the user 1. More specifically, the neural network model 10 acquired by the computing device 100 may be a model pre-trained based on a preset training dataset. In this case, the computing device 100 may update the neural network model 10 to suit the user 1 by retraining it based on the new data acquired from the user 1.

In this case, updating the neural network model 10 may involve correcting or rectifying the neural network model 10 based on the bio-information acquired by the computing device 100. In particular, this may be a process of personalizing the neural network model 10 to suit the user. For example, the computing device 100 may perform a training process of fine-tuning the neural network model 10 based on the acquired bio-information.

For example, referring to FIG. 1, when the neural network model 10 is a model trained to identify second bio-information based on first bio-information, the computing device 100 may update the neural network model 10 by retraining it based on the first bio-information acquired from the user 1. More specifically, in the case where the first bio-information is an electrocardiogram signal and the second bio-information is left ventricular ejection fraction, when an electrocardiogram signal of the user 1 is acquired, the computing device 100 may input the acquired electrocardiogram signal to the neural network model 10 to identify the left ventricular ejection fraction of the user, and may train the neural network model 10 based on the acquired electrocardiogram signal to update the neural network model 10 to suit the user 1. By repeating this process, the computing device 100 may acquire the personalized neural network model 10 (i.e., the user-customized neural network model 10) that more accurately identifies the second bio-information (e.g., left ventricular ejection fraction) of the user 1 based on the first bio-information (e.g., an electrocardiogram signal) of the user 1.

Meanwhile, referring to FIG. 1, the computing device 100 may also acquire second bio-information measured from the user. In this case, the second bio-information may be acquired from an external computing device. That is, the second bio-information may be acquired directly from the user 1 by the external computing device, or may be a value calculated based on a biosignal acquired from the user 1 by the external computing device. The second bio-information acquired from the external computing device is different from the second bio-information acquired by inputting the first bio-information to the neural network model 10. The second bio-information acquired from the external computing device may be more accurate than the second bio-information output from the neural network model 10 in that the former second bio-information is information directly measured from the user 1. The computing device 100 may also update the neural network model 10 based on the first bio-information acquired by directly measuring the user 1 by the computing device 100 together with the acquired second bio-information. In particular, the acquired second bio-information may become labeled training data, so that the computing device 100 can update the neural network model 10 to more accurately identify the second bio-information for the user 1 by using the first and second bio-information. For example, when the computing device 100 acquires a left ventricular ejection fraction value for the user 1 from an external device, the computing device 100 may update the neural network model 10 with the acquired left ventricular ejection fraction value and the electrocardiogram signal of the user corresponding to the acquired left ventricular ejection fraction.

In the following description, one embodiment of the present disclosure will be described in detail with reference to FIGS. 2 to 6.

FIG. 2 is a block diagram of a computing device 100 according to one embodiment of the present disclosure.

Referring to FIG. 2, the computing device 100 according to one embodiment of the present disclosure may include at least one processor (hereinafter, the “processor”) 110, memory 120, a network unit 130, and a sensing unit 140. However, FIG. 1 is merely an example, and the computing device 100 may further include other components for implementing a computing environment. Furthermore, only some of the disclosed components may be included in the computing device 100.

The processor 110 according to one embodiment of the present disclosure may be understood as a constituent unit including hardware and/or software for performing computing operations. For example, the processor 110 may read a computer program and perform data processing for machine learning. The processor 110 may process operation processes such as the processing of input data for machine learning, the extraction of features for machine learning, and the computation of errors based on backpropagation. The processor 110 for performing such data processing may include a central processing unit (CPU), a general purpose graphics processing unit (GPGPU), a tensor processing unit (TPU), an application specific integrated circuit (ASIC), and a field programmable gate array (FPGA). Since the types of processor 110 described above are only examples, the type of processor 110 may be configured in various manners within a range understandable to those skilled in the art based on the content of the present disclosure.

The processor 110 is electrically connected to other components (i.e., the memory 120, network unit 130, and sensing unit 140) of the computing device 100, and controls the overall operation of the computing device 100.

The memory 120 according to one embodiment of the present disclosure may be understood as a constituent unit including hardware and/or software for storing and managing data that is processed in the computing device 100. That is, the memory 120 may store any type of data generated or determined by the processor 110 and any type of data received by the network unit 130. For example, the memory 120 may include at least one type of storage medium of a flash memory type, hard disk type, multimedia card micro type, and card type memory, random access memory (RAM), static random access memory (SRAM), read-only memory (ROM), electrically erasable programmable read-only memory (EEPROM), programmable read-only memory (PROM), magnetic memory, a magnetic disk, and an optical disk. Furthermore, the memory 120 may include a database system that controls and manages data in a predetermined system. Since the types of memory 120 described above are only examples, the type of memory 120 may be configured in various manners within a range understandable to those skilled in the art based on the content of the present disclosure.

The memory 120 may structure, organize, and manage the data required for the processor 110 to perform operations, combinations of the data, and program codes executable by the processor 110. For example, the memory 120 may store a neural network model 10 trained to identify second bio-information based on first bio-information. Furthermore, the memory 120 may store program codes configured to operate the neural network model 10 to perform training based on acquired first and second bio-information, program codes configured to operate the neural network model 10 to receive time-series data and perform inference in accordance with the purpose of use of the computing device 100, and processed data that is generated as the program codes are executed.

The network unit 130 according to one embodiment of the present disclosure may be understood as a constituent unit that transmits and receives data via any type of known wired/wireless communication system. For example, the network unit 130 may perform data transmission and reception by using a wired/wireless communication system such as a local area network (LAN), a wideband code division multiple access (WCDMA) network, a long term evolution (LTE) network, the wireless broadband Internet (WiBro), a 5th generation mobile communication (5G) network, an ultra-wideband wireless communication network, a ZigBee network, a radio frequency (RF) communication network, a wireless LAN, a wireless fidelity network, a near field communication (NFC) network, a Bluetooth network, or the like. Since the above-described communication systems are only examples, the wired/wireless communication system for the data transmission and reception of the network unit 130 may be applied in various manners other than the above-described examples.

The network unit 130 may receive the data required for the processor 110 to perform operations through wired/wireless communication with any system, any client, or the like. Furthermore, the network unit 130 may transmit the data generated through the operations of the processor 110 through wired/wireless communication with any system, any client, or the like. For example, the network unit 130 may receive medical data through communication with a database within a hospital environment, a cloud server for performing tasks such as the standardization of medical data, a computing device, or the like. The network unit 130 may transmit the output data of the neural network model 10, the intermediate data derived from the computational process of the processor 110, and processed data through communication with the above-described database, server, or computing device, or the like. As an example, the processor 110 may acquire the neural network model 10 trained to identify second bio-information based on first bio-information from an external computing device (e.g., an external server device) via the network unit 130. Furthermore, the processor 110 may acquire second bio-information of the user 1 from an external computing device (e.g., a biosignal measurement device) via the network unit 130.

The sensing unit 140 may acquire first bio-information for the user 1. In particular, the sensing unit 140 may acquire first bio-information for the user 1 by using a non-invasive method. That is, according to one embodiment of the present disclosure, the first bio-information may be bio-information measured according to a non-invasive method. For example, the sensing unit 140 may include a plurality of electrodes. In this case, the processor 110 may acquire an electrocardiogram signal of the user 1 as the first bio-information via at least one electrode. Furthermore, the sensing unit 140 may include an image sensor or an optical sensor. In this case, the processor 110 may acquire a photoplethysmogram signal of the user 1 as the first bio-information via the image sensor (or the optical sensor).

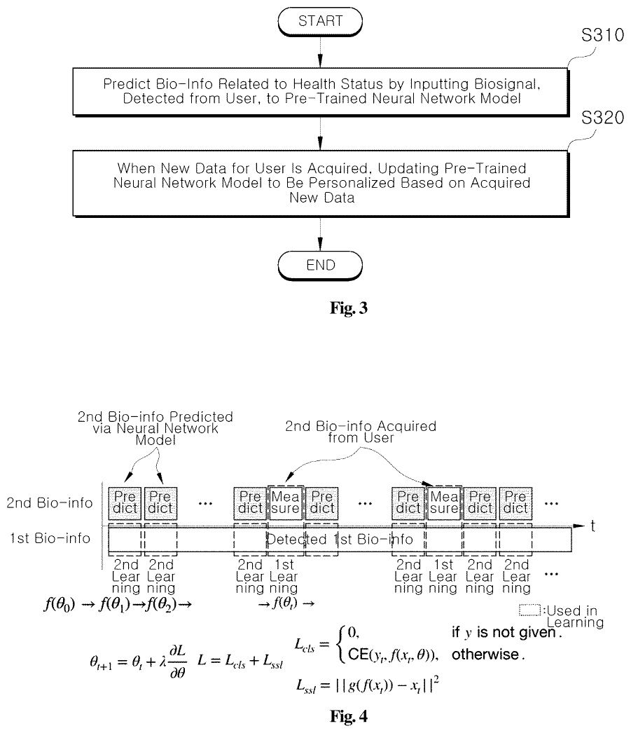

FIG. 3 is a flowchart schematically showing a method of acquiring a user-customized neural network model 10 that identifies bio-information according to one embodiment of the present disclosure.

Referring to FIG. 3, first, the processor 110 predicts bio-information related to the status of the user by inputting a biosignal, detected from the user, to the pre-trained neural network model 10 in step S310.

The processor 110 may acquire the neural network model 10 stored in the memory 120. In this case, the neural network model 10 may be stored in the memory 120 at the time when the computing device 100 is manufactured.

In this case, a plurality of neural network models 10 may be stored in the memory 120 of the computing device 100. More specifically, a plurality of neural network models 10 corresponding to a plurality of users 1 (e.g., patients, or the like) may be pre-stored in the memory 120. The processor 110 may select and acquire a neural network model 10 corresponding to the user 1 from among the plurality of neural network models 10 based on the identification information (e.g., the name, resident registration number, or like) (or the bio-information) of the user 1. For example, when the computing device 100 is a biosignal measurement device installed at a specialized medical institution, the computing device 100 may store a plurality of neural network models 10 corresponding to a plurality of patients that have visited the specialized medical institution. Each of the neural network models 10 may be repeatedly updated based on the first and second bio-information acquired for each patient and stored in the memory 120 according to an embodiment of the present disclosure to be described below.

Meanwhile, the processor 110 may also acquire the neural network model 10 from an external computing device 200 (e.g., a server device, or the like) that operates in conjunction with the computing device 100 via the network unit 130.

In this case, the external computing device 200 may be a server device that monitors the status (i.e., the disease and health status) of the user 1 of the computing device 100 while operating in conjunction with the computing device 100. Alternatively, the external computing device 200 may be a biosignal measurement device that measures second bio-information of the user 1.

Furthermore, the processor 110 may detect first bio-information for the user via the sensing unit 140. As an example, the processor 110 may detect and acquire a biosignal as the first bio-information for the user via the sensing unit 140. In this case, the biosignal may be an electrocardiogram (electrocardiogram) signal. When a command requesting the prediction of the left ventricular ejection fraction of the user 1 is input, the processor 110 may detect and acquire an electrocardiogram signal for the user via the sensing unit 140.

When first bio-information is acquired, the processor 110 may predict second bio-information by inputting the first bio-information to the neural network model 10. In particular, the second bio-information may be related to the status of the user 1. In this case, the status of the user 1 may be a disease or health status of the user. In particular, the disease or health status of the user 1 may be related to the heart of the user 1. The neural network model 10 may be a model trained to predict second bio-information based on first bio-information, as described with reference to FIG. 1. For example, when the first bio-information is an electrocardiogram signal and the second bio-information is left ventricular ejection fraction, the neural network model 10 may be trained based on a plurality of training datasets in which electrocardiogram signals and left ventricular ejection fractions corresponding to the electrocardiogram signals are matched to each other. Furthermore, the processor 110 may predict the left ventricular ejection fraction of the user 1 related with heart failure with reduced left ventricular ejection fraction by inputting the electrocardiogram signal detected from the user 1 to the neural network model 10.

Referring again to FIG. 3, according to one embodiment of the present disclosure, when new data for the user is acquired, the processor 110 may update the pre-trained neural network model to personalize it based on the acquired new data in step S320. In this case, the new data may include the bio-information measured from the user 1 in connection with the status (e.g., the disease and health status) of the user. For example, the new data may be the left ventricular ejection fraction measured from the user in relation to heart failure with reduced left ventricular ejection fraction.

The processor 110 may update the neural network model 10 by acquiring second bio-information for the user 1 and performing first learning on the neural network model 10 based on the acquired second bio-information and corresponding first bio-information.

More specifically, the processor 110 may receive the second bio-information from the external computing device 200 via the network unit 130. Alternatively, the processor 110 may receive the second bio-information via a user interface. In this case, while the processor 110 detects and acquires the first bio-information for the user 1, it may acquire the second bio-information from the external computing device 200 via the network unit 130 or acquire the second bio-information via the user interface. The second bio-information is the bio-information acquired by directly measuring the user 1, and is different from, and may be more accurate than, the second bio-information predicted by inputting the first bio-information to the neural network model 10.

As an example, the second bio-information may be the left ventricular ejection fraction measured for the user 1 by the external computing device 200. More specifically, the external computing device 200 may measure left ventricular ejection fraction by observing the internal structure of the heart of the user through an ultrasound examination and thus assessing the size and shape of the left ventricle of the user. However, the measurement of left ventricular ejection fraction is not limited thereto, and the external computing device 200 may measure the left ventricular ejection fraction of the user 1 based on various methods such as Doppler measurement, red wave emission tracing, etc. The left ventricular ejection fraction for the user 1 may be measured by a medical professional by using the external computing device 200.

Meanwhile, as another example, the second bio-information may include bio-information acquired from the user 1 by using an invasive measurement method or bio-information acquired through direct measurement by a medical professional.

Once the second bio-information has been acquired, the processor 110 may update the neural network model 10 by performing second learning on the neural network model 10 based on the acquired second bio-information. That is, the processor 110 may calibrate the pre-trained neural network model 10 to suit (or be personalized for) the user 1 based on the second bio-information.

More specifically, when the second bio-information is acquired, the processor 110 identifies the first bio-information (hereinafter, the “first bio-information corresponding to the second bio-information”) acquired via the sensing unit 140 at the time when the second bio-information is acquired (more specifically, at the time when the second bio-information is acquired by the external computing device 200), and performs second learning on the neural network model 10 by using the first and second bio-information. For example, when the first bio-information is an electrocardiogram signal and the second bio-information is left ventricular ejection fraction, the processor 110 may identify the electrocardiogram signal detected from the user 1 at the time when the external computing device 200 measures left ventricular ejection fraction of the user 1, and may perform first learning on the neural network model 10 by using the left ventricular ejection fraction acquired from the external computing device 200 and the identified electrocardiogram signal.

According to one embodiment of the present disclosure, the first learning may be supervised learning based on acquired second bio-information and first bio-information acquired to correspond to the acquired second bio-information. More specifically, the processor 110 may set the first bio-information corresponding to the second bio-information as input data and the second bio-information as a label to train the neural network model 10 on the relationship between the first and second bio-information.

Meanwhile, according to one embodiment of the present disclosure, the processor 110 may continuously acquire first bio-information for the user 1. Furthermore, the processor 110 may update the neural network model 10 by performing second learning on the neural network model 10 based on the acquired first bio-information.

More specifically, the processor 110 may continuously acquire first bio-information for the user 1 via the sensing unit 140. For example, the processor 110 may set a plurality of sample points (e.g., 125 points or 250 points) over a preset period of time (e.g., 1 second) and continuously acquire first bio-information for the user 1 across the plurality of sample points. When the first bio-information is an electrocardiogram signal, the processor 110 may acquire the electrocardiogram signal by measuring it at a sampling rate of 500 Hz for 10 seconds via the sensing unit 140. In this case, the processor 110 may process the electrocardiogram signal by removing the one-second period where the electrocardiogram signal begins and the last one-second period where the electrocardiogram signal ends in order to remove noise or artifacts contained in the electrocardiogram signal.

Furthermore, the processor 110 may continuously acquire first bio-information of the user 1 via the sensing unit 140, and may acquire a plurality of pieces of first bio-information by dividing the continuous first bio-information based on the set sample points.

Meanwhile, the processor 110 may periodically acquire first bio-information from the external computing device 200. For example, when the computing device 100 is implemented as a server device, the processor 110 may continuously receive first bio-information acquired from an external biosignal measurement device that comes into contact with the body of the user 1 and measures and acquires the first bio-information. In this case, the processor 110 may receive the first bio-information from the external biosignal measurement device via the network unit 130.

When first bio-information is acquired, the processor 110 may update the neural network model 10 by performing second learning on the neural network model 10 based on the acquired first bio-information of the user 1. That is, the processor 110 may calibrate the pre-trained neural network model 10 to suit the user 1 based on the first bio-information.

For example, when the processor 110 periodically acquires first bio-information, the processor 110 may perform second learning on the neural network model 10 by using the acquired first bio-information each time the first bio-information is acquired. That is, the second learning may be performed on the neural network model 10 in accordance with the cycle at which the first bio-information is acquired.

According to one embodiment of the present disclosure, the second learning may be self-supervised learning based on the acquired first bio-information. More specifically, the processor 110 may extract latent features from the acquired first bio-information, may assign a virtual label to the first bio-information based on the extracted latent features, and may then train the neural network model 10 based on the first bio-information assigned with the virtual label.

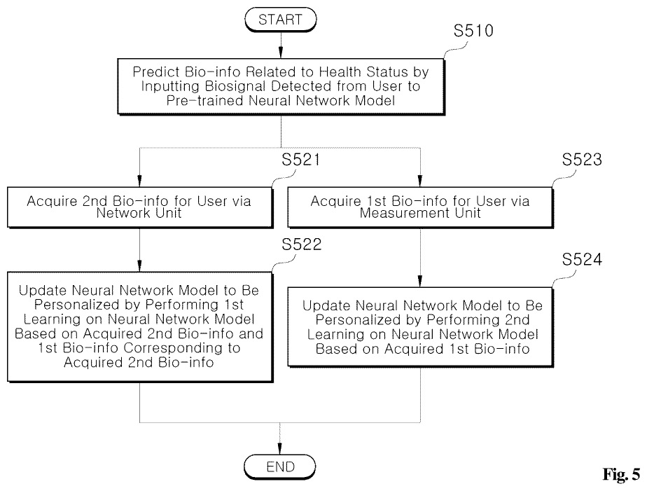

FIG. 4 is an exemplary diagram showing updating a neural network model 10 based on first and second bio-information according to one embodiment of the present disclosure. FIG. 5 is a flowchart showing updating a neural network model 10 based on first and second bio-information according to one embodiment of the present disclosure.

Meanwhile, referring to FIG. 4, for example, the first learning may be performed each time second bio-information is acquired, and the second learning may be performed each time first bio-information is acquired. In this case, when the second bio-information is acquired, the processor 110 may perform only the first learning for the neural network model 10 without performing the second learning. The reason for this is to increase the accuracy of the neural network model 10 by using the same first bio-information only for the first learning, which enables more accurate calibration of the neural network model 10, rather than using it for separate types of learning (the first and second learning). However, the learning is not limited thereto.

Meanwhile, referring to FIG. 5, step S510 shown in FIG. 5 may correspond to step S310 shown in FIG. 3. Meanwhile, referring to FIG. 5, the processor 110 may perform first and second learning processes in parallel in steps S522 and S524 as the first and second biometric information are acquired in steps S521 and S523, respectively.

In this case, the processor 110 may perform second learning on the neural network model 10 by using the periodically acquired first bio-information of the user 1 and perform first learning on the neural network model 10 by using the acquired second bio-information based on Equations 1 to 3 below. More specifically, the processor 110 may update the neural network model 10 based on gradient descent according to Equations 1 to 3 below. In this case, the processor 110 may train the neural network model 10 by using the data of time step t with a batch size of 1.

$${θ}_{t + 1} = {θ}_{t} + \frac{\partial L}{\partial θ} , L = {L}_{cls} + {L}_{ssl} \qquad ( 1 )$$

$${L}_{cls} = \{ \begin{matrix}0 , & if y is not given \\ CE ( {y}_{t} , f ( {x}_{t} , θ ) ) , & otherwise\end{matrix} \qquad ( 2 )$$

$${L}_{ssl} = {‖ g ( f ( {x}_{t} ) ) - {x}_{t} ‖}^{2} \qquad ( 3 )$$

In this case, θₜ may be a parameter of the neural network model at time t. xₜ may be the first bio-information acquired at time t, and yₜ may be the second bio-information acquired at time t. L may be the overall loss function of the neural network model, L_{cls} may be a supervised learning loss function, and Lₛₛₗ may be a self-supervised learning loss function. L_{cls} is defined as the cross-entropy loss CE(yₜ, f(xₜ,θ)) between the second bio-information predicted based on the first bio-information acquired at time t and the second bio-information actually acquired at time t, and Lₛₛₗ may be defined as a loss function that minimizes the Euclidean distance between g(f(xₜ)), i.e., the result of the embedding of f(xₜ), and the input data xₜ.

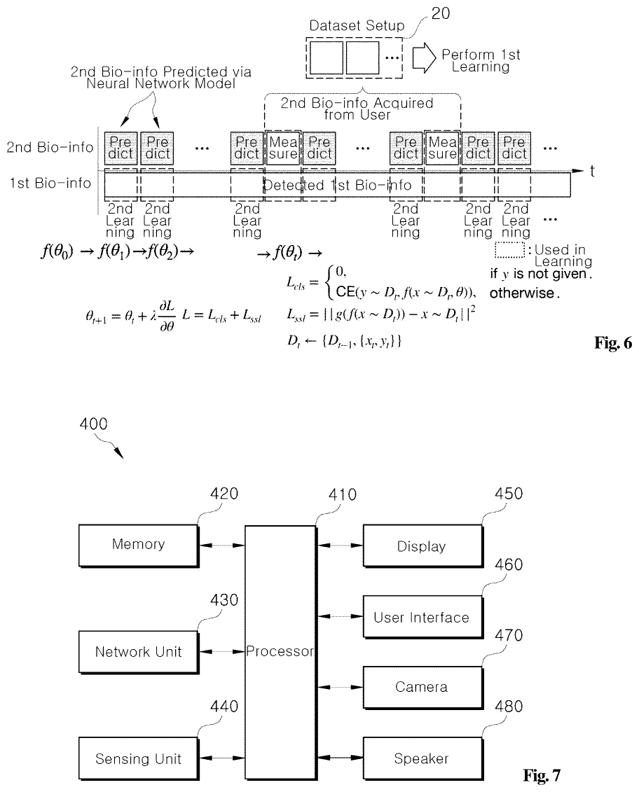

FIG. 6 is an exemplary diagram showing updating the neural network model 10 based on first bio-information and a plurality of pieces of second bio-information included in a dataset according to one embodiment of the present disclosure.

Meanwhile, according to one embodiment of the present disclosure, when new data for a user is acquired, the processor 110 may add the acquired new data to the dataset accumulated and acquired at a previous time, and may perform first learning on the neural network model 10 based on the dataset, thereby updating the neural network model to be personalized. In this case, the dataset may include at least one second bio-information accumulated and acquired at a previous time and first bio-information corresponding to the at least one second bio-information, which are matched with each other.

More specifically, referring to FIG. 6, when new second bio-information is acquired, the processor 110 may identify the first bio-information acquired at the time when the second bio-information is acquired and add the second bio-information and the first bio-information to a dataset 20 accumulated and acquired at a previous time. In this case, the dataset 20 may include a plurality of pieces of second bio-information accumulated and acquired before the time when the new second bio-information is acquired, and a plurality of pieces of first bio-information corresponding to the plurality of pieces of second bio-information. That is, when the second bio-information is acquired, the processor 110 may update the dataset 20 with the acquired second bio-information and the first bio-information corresponding to the acquired second bio-information. Furthermore, the processor 110 may perform first learning on the neural network model 10 based on the dataset 20, thereby updating the neural network model 10.

In this case, the processor 110 may perform second learning on the neural network model 10 by using the periodically acquired first bio-information of the user 1 and perform first learning on the neural network model 10 by using the dataset 20 including the acquired plurality of pieces of second bio-information based on Equations 4 to 7 below. More specifically, the processor 110 may update the neural network model 10 based on gradient descent according to Equations 4 to 7 below:

$${θ}_{t + 1} = {θ}_{t} + \frac{\partial L}{\partial θ} , L = {L}_{cls} + {L}_{ssl} \qquad ( 4 )$$

$${L}_{cls} = \{ \begin{matrix}0 , & if y is not given \\ CE ( {y}_{t} ∼ {D}_{t} , f ( {x}_{t} ∼ {D}_{t} , θ ) ) , & otherwise\end{matrix} \qquad ( 5 )$$

$${L}_{ssl} = {‖ g ( f ( {x}_{t} ∼ {D}_{t} ) ) - {x}_{t} ∼ {D}_{t} ‖}^{2} \qquad ( 6 )$$

$${D}_{t} ← \{ {D}_{t - 1} , \{ {x}_{t} , {y}_{t} \} \} \qquad ( 7 )$$

In this case, θ, may be a parameter of the neural network model at time t. xₜ may be the first bio-information acquired at time t, and yₜ may be the second bio-information acquired at time t. L may be the overall loss function of the neural network model, L_{cls} may be a supervised learning loss function, and Lₛₛₗ may be a self-supervised learning loss function. L_{cls} is defined as the cross-entropy loss CE(yₜ,f(xₜ,θ)) between the second bio-information predicted based on the first bio-information acquired at time t and the second bio-information actually acquired at time t, and Lₛₛₗ may be defined as a loss function that minimizes the Euclidean distance between g(f(xₜ)), i.e., the result of the embedding of f(xₜ), and the input data xₜ. Dₜ may be the dataset 20.

Meanwhile, the processor 110 may identify the bio-information of the user 1, and may share the dataset 20 related to second bio-information with another computing device 100 of another user 1 having bio-information similar to the identified bio-information. In this case, the bio-information may include the height, weight, age, gender, and/or the like of the user 1. For example, when the computing device 100 is a smartwatch, the processor 110 may acquire the bio-information of another user 1 from another smartwatch information of the other user 1. The processor 110 may identify the similarity between the bio-information of a plurality of other users 1 and the bio-information of the user 1, and may identify another computing device 100 of another user 1 having bio-information similar to the bio-information of the user 1 based on the identified similarity. Furthermore, the processor 110 may share the dataset 20 related to the second bio-information by transferring the acquired dataset 20 to the identified computing device 100. Alternatively, the processor 110 may also have the dataset 20 related to the second bio-information of another user 1 shared from another computing device 100 of the other user 1 having bio-information similar to that of the user 1. In particular, the second bio-information is acquired intermittently because it is measured non-invasively or by a medical professional, and the amount of data may be small. Accordingly, in the case of a plurality of users 1 having similar bio-information, the neural network model 10 may be updated to more rapidly reflect the characteristics of the user 1 therein by sharing the dataset 20 related to acquired second bio-information.

As an example, a server device operating in conjunction with the computing device 100 may acquire bio-information of the user 1 from the computing device 100, and may transmit the neural network model 10 corresponding to the acquired bio-information to the computing device 100. More specifically, the server device may store a plurality of neural network models 10 corresponding to a plurality of users 1. In particular, each of the neural network models 10 may correspond to a user (1) group including a plurality of users 1 having similar bio-information. Accordingly, when bio-information of the user 1 is received from the computing device 100, the server device may select bio-information similar to the received bio-information and transmit the neural network model 10 corresponding to the selected bio-information to the computing device 100. In this case, the server device may also include the user 1 of the computing device 100 in the user (1) group corresponding to the selected bio-information. The server device may share second bio-information acquired from each computing device 100 (or the dataset 20 set based on the second bio-information) for a plurality of users 1 included in the same user (1) group.

According to one embodiment of the present disclosure, the processor 110 may identify bio-information of the user 1, may cluster the user 1 and a plurality of other users 1 based on the identified bio-information, and may assign the dataset 20 including new data to each clustering group. More specifically, the processor 110 may acquire bio-information of a plurality of users 1 from a plurality of user terminal devices that operates in conjunction with the computing device 100. In this case, the processor 110 may cluster a plurality of users 1 based on bio-information of the plurality of users 1. Furthermore, the processor 110 may set the dataset 20 for each clustering group by using the second biosignals acquired from the user terminal devices included in each clustering group. That is, when second biosignals are acquired from each of multiple user (1) terminal devices included in a specific clustering group, the processor may collect the plurality of acquired biosignals, may set up the dataset 20 for the specific clustering group, and may assign the dataset 20 to the specific clustering group. In this case, the computing device 100 may be implemented as a server device that manages the status (e.g., the disease and health status) of the plurality of users 1.

According to one embodiment of the present disclosure, the processor 110 may predict second bio-information for the user 1 by inputting the acquired first bio-information of the user 1 to the neural network model 10. This may be performed through the second learning (i.e., self-supervised learning) process described above. Furthermore, the processor 110 monitors the trend of predicted second bio-information, and may acquire second bio-information from the external computing device 200 when a change in the trend of predicted second bio-information is detected. Alternatively, the processor 110 may acquire the second bio-information acquired from the external computing device 200 and stored in memory. Furthermore, the processor 110 may update the neural network model 10 by performing first learning on the neural network model 10 based on the acquired second bio-information. That is, when a change in the trend or tendency of second bio-information is detected, the processor 110 may determine to update the neural network model 10 based on the second bio-information. For example, when it is determined that the similarity between the trend of second bio-information in a previous cycle and the trend of second bio-information in a current cycle is less than a preset value, the processor 110 may identify that the trend of second bio-information has changed and determine to perform first learning on the neural network model 10 by using the second bio-information.

Furthermore, according to one embodiment of the present disclosure, when it is determined that a preset time has elapsed since a previous update time, the processor 110 may update a pre-trained neural network model based on acquired new data so that it can be personalized. In this case, the previous update time may be the time when the most recent update was performed. That is, when it is determined that a preset time has elapsed since the most recent update time, the processor 110 may determine to perform the process of updating the pre-trained neural network model 10. For example, assuming that the preset time is 7 days, the processor 110 may perform the process of updating the neural network model 10 when it is determined that 10 days have elapsed since the most recent update time.

Furthermore, according to one embodiment of the present disclosure, when the number of times bio-information related to the status of the user has been predicted since the previous update time is equal to or larger than a preset value, the processor 110 may update the pre-trained neural network model 10 based on the acquired new data so that it can be personalized. In this case, the previous update time may be the most recent update time. For example, assuming that the preset value of 20 times, the processor 110 may perform the process of updating the neural network model 10 when the number of times second bio-information (e.g., left ventricular ejection fraction) has been predicted via the neural network model 10 since the most recent update time is identified as 25 times.

Meanwhile, when it is identified that a preset amount of time has elapsed since the previous update time, the processor 110 may identify whether the number of predicted bio-information related to the user's status from the previous update is equal to or larger than a preset value and determine whether to perform the process of updating the neural network model 10.

According to one embodiment of the present disclosure, when second bio-information is acquired, the processor 110 may acquire second bio-information as output data by inputting first bio-information corresponding to the second bio-information to the neural network model 10, and may identify the error between the acquired second bio-information and the output data. When the error between the second bio-information acquired as the output data and the second bio-information acquired from another computing device 100 is smaller than a preset value, the processor 110 may determine to stop the process of updating the neural network model 10 (e.g., a first learning process). The reason for this is to determine that the process of personalizing the neural network model 10 for the user 1 is complete, thereby reducing the resources of the computing device 100 required for learning.

Furthermore, according to one embodiment of the present disclosure, when it is determined based on acquired first bio-information that the user 1 has changed, the processor 110 may stop the update process, may replace the neural network model 10 with a new neural network model, and may resume the update process.

According to one embodiment of the present disclosure, when the first bio-information includes electrocardiogram (ECG) information and the second bio-information includes left ventricular ejection fraction (EF), the processor 110 may identify the left ventricular ejection fraction of the user 1 by inputting the electrocardiogram information to the neural network model 10, and may predict heart failure with reduced left ventricular ejection fraction in the user 1 based on the identified left ventricular ejection fraction. For example, the processor 110 may calculate a score corresponding to the left ventricular ejection fraction of the user 1 identified via the neural network model 10, and may identify the latent possibility of heart failure with reduced left ventricular ejection fraction in the user 1 based on the calculated score. In this case, when the left ventricular ejection fraction is lower than a preset value or the score is equal to or higher than a preset value, the processor 110 may identify the possibility of heart failure with reduced left ventricular ejection fraction as being at a risky level, and may output a warning message, and/or the like via an output interface (e.g., a speaker, a display, and/or the like).

FIG. 7 is a block diagram of a computing device 400 according to another embodiment of the present disclosure.

Referring to FIG. 7, the computing device 400 includes a processor 410, memory 420, a network unit 430, a sensing unit 440, a display 450, a user interface 460, a camera 470, and a speaker 480. Among the components shown in FIG. 7, the processor 410, the memory 420, the network unit 430, and the sensing unit 440 correspond to the processor 110, memory 120, network unit 130, and sensing unit 140 of the computing device 100 shown in FIG. 2, so that detailed descriptions thereof will be omitted.

The display 450 may display various images. The images include both still images and moving images. The display 450 may output activity-related guidance information generated based on the status of the user 1. The display 450 may be implemented in the form of various types of displays, such as an liquid crystal display (LCD) panel, an organic light emitting diode (OLED) display, a liquid crystal on silicon (LCoS) display, a digital light processing (DLP) display, and the like. Furthermore, the display 450 may also include a driving circuit, a backlight unit, and the like, which can be implemented in the form of elements such as a-Si TFTs, low temperature poly silicon (LTPS) TFTs, and organic TFTs (OTFTs).

Meanwhile, the display 450 may be implemented as a touch screen in combination with a touch panel. In this case, the display 450 may function not only as an output interface configured to output images via the touch screen, but also as an input interface configured to receive touch input from the user 1.

The user interface 460 is a component used by the computing device 100 to interact with the user 1. The user interface 460 may include, but is not limited to, at least one of a touch sensor, a motion sensor, buttons, a jog dial, and switches. The processor 410 may receive second bio-information and the bio-information (e.g., the occupation, age, and gender) of the user 1 via the user interface 460.

The camera 470 acquires an image of an object near the user 1 by capturing the image of the object More specifically, the camera 470 may capture an image of food consumed by the user 1. In this case, the processor 410 may determine the nutritional status of the user 1 and provide recommended meal information as guidance information based on the status of the user 1 and the image of the food consumed. For this purpose, the camera 470 may be implemented as an imaging device such as a CMOS image sensor (CIS) having a CMOS structure or a charge-coupled device (CCD) having a CCD structure. However, the camera 470 is not limited thereto, and may be implemented as camera modules having various resolutions capable of capturing subjects. Meanwhile, the camera 470 may be implemented as a depth camera (e.g., an IR depth camera), a stereo camera, or an RGB camera.

The speaker 480 is a component that outputs various audio data that has undergone various processing operations, such as decoding, amplification, and noise filtering, via an audio processing unit (not shown). The speaker 480 may output various types of notification sounds or voice messages. According to one embodiment of the present disclosure, the processor 410 may convert an electrical signal received from an external device into the voice of the user 1 and output it via the speaker 480. For example, the speaker 480 may output a voice message warning of a disease or suggesting a diagnosis of a disease related with the second bio-information identified via the neural network model 10.

The various embodiments of the present disclosure described above may be combined with one or more additional embodiments, and may be changed within the range understandable to those skilled in the art in light of the above detailed description. The embodiments of the present disclosure should be understood as illustrative but not restrictive in all respects. For example, individual components described as single may be implemented in a distributed manner, and similarly, the components described as distributed may also be implemented in a combined form. Accordingly, all changes or modifications derived from the meanings and scopes of the claims of the present disclosure and their equivalents should be construed as being included in the scope of the present disclosure.

1. A method of acquiring a user-customized neural network model that identifies bio-information, the method being performed by a computing device including at least one processor, the method comprising:
predicting bio-information related to a status of a user by inputting a biosignal, detected from the user, to a pre-trained neural network model; and
based on new data for the user being acquired, updating the pre-trained neural network model to be personalized based on the acquired new data;
wherein the new data comprises bio-information measured from the user related to the status.

2. The method of claim 1, wherein the updating comprises calibrating the pre-trained neural network model to be personalized based on the measured bio-information and the biosignal detected to correspond to the measured bio-information.

3. The method of claim 2, wherein the calibration comprises performing supervised learning based on the bio-information measured from the user and the biosignal detected to correspond to the bio-information measured from the user.

4. The method of claim 1, wherein:
the updating comprises: accumulating the acquired new data into a dataset accumulated and acquired at a previous time based on new data for the user being acquired, and updating the neural network model to be personalized based on the dataset; and
the dataset comprises a plurality of pieces of bio-information accumulated and measured at the previous time from the user.

5. The method of claim 1, comprising identifying bio-information of the user, and transmitting the new data to a computing device of another user having bio-information similar to the identified bio-information so that the computing device of the other user is updated based on the new data.

6. The method of claim 1, wherein:
the biosignal comprises an electrocardiogram (ECG) signal; and
the bio-information comprises left ventricular ejection fraction (EF).

7. The method of claim 6, comprising, when the left ventricular ejection fraction is lower than a preset value, outputting information warning of heart failure with reduced left ventricular ejection fraction of the user.

8. The method of claim 1, wherein the updating comprises monitoring a trend of the predicted bio-information, and, updating the pre-trained neural network model to be personalized based on the acquired new data based on when a change in the predicted bio-information is detected.

9. The method of claim 1, wherein the updating comprises, when it is identified that a preset time has elapsed since the previous update time, updating the pre-trained neural network model to be personalized based on the acquired new data.

10. The method of claim 1, wherein the updating step comprises, when a number of times bio-information related to the status of the user has been predicted since the previous update time is equal to or larger than a preset value, updating the pre-trained neural network model to be personalized based on the acquired new data.

11. A computer program stored in a computer-readable storage medium, the computer program causing operations for acquiring a user-customized neural network model that identifies bio-information to be performed when executed by at least one processor, wherein the operations comprise operations of:
predicting bio-information related to a status of a user by inputting a biosignal, detected from the user, to a pre-trained neural network model; and
based on new data for the user being acquired, updating the pre-trained neural network model to be personalized based on the acquired new;
wherein the new data comprises bio-information measured from the user related to the status.

12. A computing device for acquiring a user-customized neural network model that identifies bio-information, the computing device comprising:
memory including program codes;
a network unit;
a sensing unit configured to detect bio-information of a user; and
a processor configured to predict bio-information related to a status of the user by inputting a biosignal, detected from the user, to a pre-trained neural network model, and to, based on new data for the user being acquired, update the pre-trained neural network model to be personalized based on the acquired new data;
wherein the new data comprises bio-information measured from the user related to the status.
